(19) United States
(12) Patent Application Publication
(10) Pub. No.: US 2012/0004710 A1
(43) Pub. Date: Jan. 5, 2012

(54) IRRADIATING STRIP CATHETER

(76) Inventor: Tom Kerber, Stoney Creek (CA)

(21) Appl. No.: 12/672,570

(22) PCT Filed: Aug. 10, 2008
(86) PCT No.: PCT/IL08/01101

§ 371 Date: Mar. 5, 2010

(30) Foreign Application Priority Data
Aug. 8, 2007 (IL) 185128

(51) Int. Cl. A61N 5/06

(52) U.S. Cl. 607/80

(57) ABSTRACT

The invention discloses a Light Irradiating Unit (LIU) for providing radiant energy safely to a treatment site within the human body during treatment sessions. the LIU is comprised of the following modules: an Irradiating Array (IA) comprising LED's longitudinally disposed in a single row along a flexible support member; an external LED Controller and Power Supply (CPS); an Array Housing Catheter (AHC) for enclosing and guiding said IA to the treatment site; and a Cooling Catheter (CC) for enclosing and cooling the AHC during treatment sessions; the CC connects to a standard intra venous bag, containing cooling fluid, the is bag adapted to provide gravity induced cooling fluid flow through the CC during treatment sessions; the CC further adapted to provide drainage of the cooling fluid during said treatment sessions.

This invention generally relates to a device and methods for providing radiant light energy safely to a treatment site within the human body during treatment sessions and more generally, the mammalian body.

### BACKGROUND

This invention generally relates to a device and method for treating sites within the human body, and more generally, the mammalian body, with radiant energy for use in photodynamic therapy. Photodynamic therapy (PDT) is increasingly used for treatment of tumours and to destroy microbes, lesions and the like. When PDT is used to treat malignant tumours, a photosensitive dye is injected into the patient at a predetermined time before the planned therapy. The photosensitive dye is activated at wavelengths which penetrate cancerous cells, microbes or other target cells, oxygen radicals are produced which destroy the target cells.

LED arrays mounted in a substantially cylindrical support to which is attached a removable multisided head are proposed in U.S. Pat. No. 5,728,090 to Martin et al, however, the non flexibility and wide diameter of the arrangement limits the type of procedures in which such a device can be used. Likewise, a semiconductor light emitting device system is proposed in US patent application 2007/0168000 to Happawana et al but it is also inflexible. Van Zuylen et al in U.S. Pat. No. 6,221,095 proposes a photon therapy unit implement design for external applications only.

Thus while the prior art has attempted to address the problem of delivering effective radiant light energy to a space within the human body, the prior art devices do not enable easy access to a wide range of internal body sites. Thus, there is still a long felt need for a device that will deliver radiant light energy to a site within the mammalian or human body while providing cooling to counteract heating effects so that the optimum safe amount of radiant energy can be administered.

In general surgery, the emphasis has been on laparoscopic techniques, which can now be applied to the majority of intra-abdominal procedures. A further long felt need would be fulfilled by providing means and method for administering radiant light energy to internal body sites during laparoscopic procedures.

There is also now a growing interest in endoluminal procedures—flexible endoscopic approaches to the treatment of disease, dispensing with incisions in the abdominal wall by using a natural orifice (i.e., the mouth) to access the target tissue. Other natural orifices, such as the anus or vagina, may also allow access to the peritoneal cavity.

Flexibility of the LED circuit boards is an issue which must be addressed; even the straight narrow flexible Mylar or Kapton circuit boards are flexible in one direction but not very flexible in another. A long felt need would be filled if circuit boards suitable for mounting LED's had good flexibility in all directions.

A yet further long felt need would therefore be fulfilled by providing means and method for administering radiant light energy to internal body sites during NOTES (natural orifice transluminal endoscopic surgery).

Ease of use, safety and compatibility of medical devices and methods with existing hospital standard equipment, practices and power sources is another long felt need to be addressed.

### SUMMARY OF THE INVENTION

It is one object of the invention to disclose a Light Irradiating Unit (LIU) for providing radiant energy safely to a treatment site within the human body during treatment sessions; wherein the LIU is comprised of the following modules: an Irradiating Array (IA) comprising a plurality of LED's longitudinally disposed in a single row along a flexible support member; the LED's are electrically connected in series to a battery powered external LED Controller and Power Supply; the IA is rotatable in situ through 360 degrees of movement; an external. LED Controller and Power Supply (CPS); an Array Housing Catheter (AHC) for enclosing and guiding said IA to the treatment site; and

a Cooling Catheter (CC) for enclosing and cooling the AHC during treatment sessions; the CC is adapted to connect to a standard intra venous bag, containing cooling fluid, the bag adapted to provide gravity induced cooling fluid flow through the CC during treatment sessions; the CC further adapted to provide drainage of the cooling fluid during said treatment sessions.

It is another object of the invention to provide an LIU as defined above wherein the CPS further comprises a battery powered cell.

It is another object of the invention to provide an LIU as defined above wherein the LIU further comprises a motor driven means of rotating at least one of the group consisting of the IA, said AHC and said CC in a predetermined manner as required by the treatment protocol.

It is another object of the invention to provide an LIU as defined above wherein the LIU additionally comprises the CPS with a rechargeable battery and a first connecting means for connecting CPS to a wall power adapter for recharging the battery. A second connecting means for connecting the CPS to the IA is included. The safety means is adapted so as to ensure that the first or second connecting means make first or second connections respectively at any one time and so arranged that the first or second connections cannot be completed or maintained contemporaneously.

It is another object of the invention to provide an LIU as defined above wherein the CPS further comprises means for operation by remote control.

It is another object of the invention to provide an LIU as defined above wherein the CC is adapted to connect to a standard hospital pump, the pump being adapted to induce cooling fluid flow during treatment sessions.

It is another object of the invention to provide an LIU as defined above wherein the LED's are electrically connected in parallel to a battery powered external LED CPS.

It is another object of the invention to provide an LIU as defined above wherein the flexible support member is fan folded.

It is another object of the invention to disclose a method of providing radiant energy safely to a treatment site within the human body during treatment sessions; the method including steps of: obtaining an Irradiating Array (IA) and disposing a plurality of LED's longitudinally in a single row along a flexible support member; the LED's are electrically connected in series to a battery powered external LED CPS; the IA is rotatable in situ through 360 degrees of movement; obtaining an external LED Controller and Power Supply (CPS); obtaining an Array Housing Catheter (AHC) for enclosing and guiding the IA to the treatment site; and, further obtaining a Cooling Catheter (CC) for enclosing and cooling the AHC during treatment sessions; the CC is adapted to connect to a standard intra venous bag, containing cooling fluid, adapting the bag to provide gravity induced cooling fluid flow through the CC during treatment sessions; further adapting the CC to provide drainage of the cooling fluid during the treatment sessions.

It is another object of the invention to disclose a method, wherein the method includes additional steps of powering the CPS by means of a battery cell.

It is another object of the invention to disclose a method wherein the method further includes obtaining a motor driven means of rotating at least one of the group consisting of the IA, the AHC and the CC in a predetermined manner as required by the treatment protocol.

It is yet another object of the invention to disclose a Method of providing radiant energy safely to a treatment site within the human body during treatment sessions, the method including additional steps of providing the CPS with a rechargeable battery, firstly connecting the CPS to a wall power adapter, secondly connecting the CPS to the IA, adapting the first connection between the wall power adapter and the CPS, and the second connection between CPS and the IA such that only one of the first or second connections can be made at any one time and recharging the CPS by means of establishing the first connection.

It is a further object of the invention to disclose a method wherein the method includes steps of operating the CPS by remote control.

It is a still further object of the invention to disclose a method wherein the method includes steps of connecting the CC is to a standard hospital pump, further adapting the pump to induce cooling fluid flow during treatment sessions.

Moreover, it is yet another object of the invention to disclose a method wherein the method includes steps of electrically connecting the LED's in parallel to a battery powered external LED CPS.

Lastly, it is another object of the invention to disclose a method wherein the method includes steps of fan folding the flexible support member.

### BRIEF DESCRIPTION OF THE DRAWINGS

FIG. 1 is a schematic drawing of the LED Controller and Power Supply (CPS).

FIG. 2 is a schematic representation of the duty cycle chart

FIG. 3 is a schematic drawing of an embodiment of the invention.

FIG. 4 is a schematic drawing of an embodiment of the invention.

FIG. 5 is a schematic drawing an embodiment of the invention illustrating a flexible circuit board “fan folded” under itself for each LED.

### DETAIL DESCRIPTION OF THE SPECIFIC EMBODIMENTS

The following description is provided, alongside all chapters of the present invention, so as to enable any person skilled in the art to make use of said invention and sets forth the best modes contemplated by the inventor of carrying out this invention. Various modifications, however, is adapted to remain apparent to those skilled in the art, since the generic principles of the present invention have been defined specifically to provide a device and methods for providing radiant energy safely to a treatment site within the human body or more generally a mammalian body during treatment sessions.

Before explaining the figures and examples, it should be understood that the invention is not limited in its application to the details of construction and the arrangement of the components set forth in the following description or illustrated in the drawings. The invention can be carried out in various ways as will be apparent to a person skilled in the art.

### Rationale for Water Cooling of the LED's In Situ.

LED's generally produce 10% light output and 90% heat output. Thus a typical LED used for photodynamic therapy may produce 1 Watt of heat, and an array of 10 LED's may produce 10 Watts of heat. If excess heat is not removed, LED's would burn out, and of course, burn injuries could be inflicted on the patient.

### Example

BTU are abbreviations for British Thermal Units. h=hours. W=weight of water or coolant.

In an operating situation, 1 litre IV bag of water or another medically acceptable coolant would be used, weighing 2.2 pounds. If the treatment session lasts 1 hour, then:

1 Watt for each LED=3 BTU of heat.
10 LED's produce 30 BTU of heat.

Since

$$Δ {T}^{0} = \frac{BTUh}{W} = 30 / 2.2 = 13.6 {F}^{0}$$

the total operating temperature inside the body will be 83.6 F (considered safe), assuming that the ambient water temperature is 70 F °.

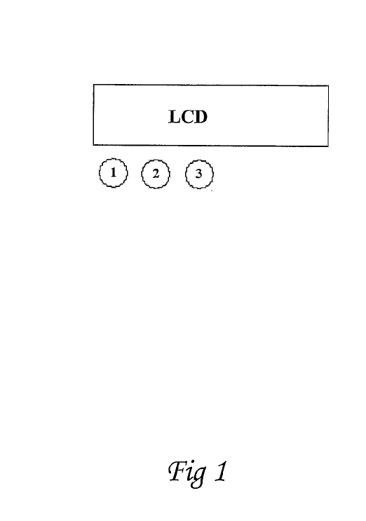

### Example: (See FIG. 1)

### LED Controller and Power Supply (CPS)

The LCD displays the value of the modes chosen.

1. Button 1 changes modes.
2. Button 2 controls up function
3. Button 3 controls down function.

The two functions of this unit is to adjust the power (brightness) given to the LED's, and to monitor the temperature of the LED's. When in the power setting display mode, current power is displayed. LED power can be adjusted by pressing button 1. To decrease power the second button is pressed. Current operating temperature is also displayed. Temperature mode displays set temperature. Maximum temperature can be altered before shutting down.

### Example

### LED Power Control

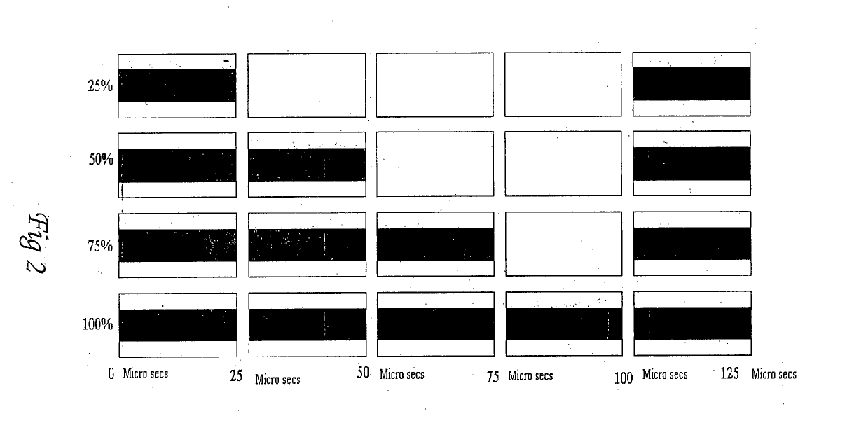

FIG. 2 is a schematic representation of the duty cycle chart. The LED power setting is controlled by the duty cycle.

When the power level is 0 the duty cycle is turned on.

At 25% power level the light is on for 25 micro seconds and off for 75 micro seconds.

At 50% power level the light is on for 50 micro seconds and off for 50 micro seconds.

At 75% power level the light is on for 75 micro seconds and off for 25 micro seconds.

At 100% power level the light is continuously on.

### Example

### Temperature Control

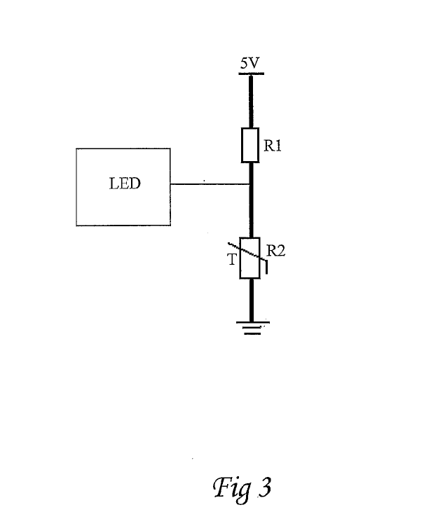

Reference is now made to FIG. 3 which is a schematic drawing of the thermistor/LED arrangement.

The temperature control is controlled by a voltage change of the resistance across ten thermistors. Each thermistor has 1 high power LED on it.

Example of resistance of various temperatures for one type of thermistor:

25 degrees C., resistance value would be 200 ohms per thermistor, totaling 2000 ohms for ten thermistors in a series.
50 degrees C., resistance value would be 100 ohms per thermistor, totaling 1000 ohms for ten thermistors in a series.
75 degrees C., resistance value would be 50 ohms per thermistor, totaling 500 ohms for ten thermistors in a series.
100 degrees C., resistance value would be 25 ohms per thermistor, totaling 250 ohms for ten thermistors in a series.

### Example of an Embodiment of the Invention

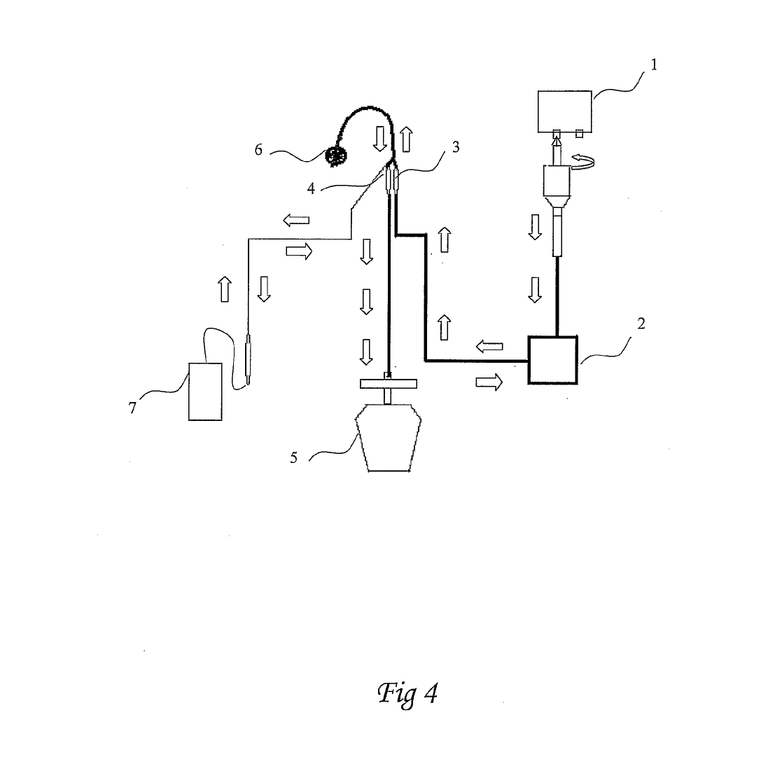

Reference is now made to FIG. 4 which is a schematic drawing of a non limiting particular embodiment of the invention;

1. cooling fluid in the IV bag is connected to the pump unit.

2. Pump unit distributes the cooling fluid to the catheter tube.

3. cooling fluid is forced through the catheter to cool the LED's

4. Cooling fluid is drained back out of the catheter.

5. Cooling fluid flows back to the drainage container from where it is discarded.

6. LED Controller sends power to control the LED's

7. LED board sends back operating conditions

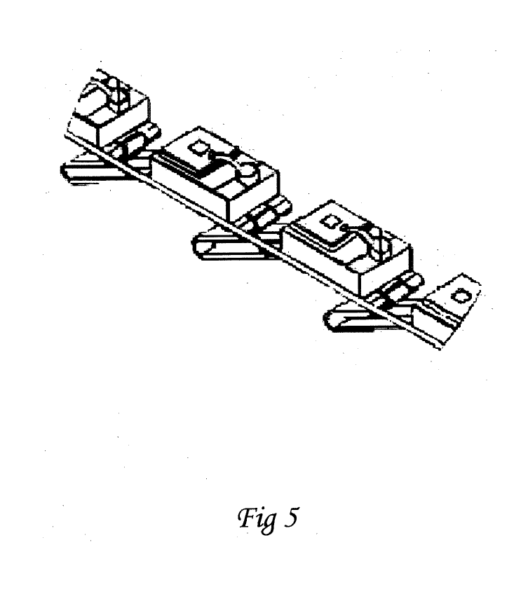

A key core embodiment of the invention is the provision of a flexible circuit board “fan folded” under itself for each LED as whole or part of the flexible member upon which the LED's are mounted longitudinally in a single row as shown in the example in FIG. 5. Such fan folding provides high flexibility in all directions while not putting mechanical stress on the parts.

It is in the scope of the invention to provide a Light Irradiating Unit (LIU) for providing radiant energy safely to a treatment site within the human body during treatment sessions. The LIU is comprised of the following modules: An Irradiating Array (IA) comprising a plurality of LED's longitudinally disposed in a single row along a flexible support member; the LED's are electrically connected in series to a battery powered external LED Controller and Power Supply; and the IA is rotatable in situ through 360 degrees of movement. An external LED Controller and Power Supply (CPAn Array Housing Catheter (AHC) which encloses and guides the IA to the treatment site; and a Cooling Catheter (CC) for enclosing and cooling the AHC during treatment sessions. The CC is adapted to connect to a standard intra venous bag, containing cooling fluid. The bag is adapted to provide gravity induced cooling fluid flow through the CC during treatment sessions and the CC is further adapted to provide drainage of the cooling fluid during the predetermined treatment sessions.

It is within the scope of the invention to provide an LIU as defined above, additionally providing the CPS with a battery powered cell.

It is within the scope of the invention to provide an LIU as defined above, additionally providing a motor driven means of rotating at least one of the IA, AHC and CC in a predetermined manner as required by the treatment protocol.

It is within the scope of the invention to provide an LIU as defined above, the LIU additionally comprising a CPS with a rechargeable battery first connecting means for connecting CPS to a wall power adapter for recharging said battery. A second connecting means for connecting said CPS to the IA is also provided. The safety means adapted so as to ensure that said first or second connecting means make first or second connections respectively at any one time and that the first or second connections cannot be completed or maintained contemporaneously.

It is within the scope of the invention to provide an LIU as defined above such that the CPS further comprises means for operation by remote control.

It is also within the scope of the invention to provide an LIU as defined above such that the CC is adapted to connect to a standard hospital pump, the pump being adapted to induce cooling fluid flow during treatment sessions.

It is also within the scope of the invention to provide an LIU as defined above such that the LED's are electrically connected in parallel to a battery powered external LED CPS.

It is also within the scope of the invention to provide an LIU as defined above such that the flexible support member is fan folded.

It is also within the scope of the invention to provide a method of providing radiant energy safely to a treatment site within the human body during treatment sessions; the method including steps of obtaining an Irradiating Array (IA) and disposing a plurality of LED's longitudinally in a single row along a flexible support member; the LED's are electrically connected in series to a battery powered external LED CPS; the IA is rotatable in situ through 360 degrees of movement; obtaining an external LED Controller and Power Supply (CPS); obtaining an Array Housing Catheter (AHC) for enclosing and guiding said IA to said treatment site; and, further obtaining a Cooling Catheter (CC) for enclosing and cooling said AHC during treatment sessions; said CC is adapted to connect to a standard intra venous bag, containing cooling fluid, adapting said bag to provide gravity induced cooling fluid flow through said CC during treatment sessions; further adapting said CC to provide drainage of said cooling fluid during said treatment sessions.

It is also within the scope of the invention to provide a method as defined above, wherein the method includes additional steps of powering the CPS by means of a battery cell.

It is also within the scope of the invention to provide a method as defined above wherein the method further includes obtaining a motor driven means of rotating at least one of the group consisting of said IA, said AHC and said CC in a predetermined manner as required by the treatment protocol.

It is also within the scope of the invention to provide a method as defined above of providing radiant energy safely to a treatment site within the human body during treatment sessions. The method includes additional steps of) providing the CPS with a rechargeable battery firstly connecting the CPS to a wall power adapter secondly connecting the CPS to the IA adapting the first connection between the wall power adapter and the CPS, and the second connection between CPS and the IA such that only one of the first or second connections can be made at any one time, and recharging the CPS by means of establishing the first connection.

It is also within the scope of the invention to provide a method as defined above wherein the method includes steps of operating CPS by remote control.

It is also within the scope of the invention to provide a method as defined above wherein the method includes steps of connecting the CC is to a standard hospital pump, further adapting the pump to induce cooling fluid flow during treatment sessions.

It is also within the scope of the invention to provide a method as defined above wherein the method includes steps of electrically connecting said LED's in parallel to a battery powered external LED CPS.

It is also within the scope of the invention to provide a method as defined above wherein the method includes steps of fan folding said flexible support member.

While the devices and methods described are for the delivery of radiant light energy for the purposes of photodynamic therapy in treatment of bladder tumours, gastrointestinal tumours, brain tumours, prostate tumours, and stomach tumours, the invention contemplates the use of similar devices properly sized and shaped for administration of similar treatments to other body organs and tissues and tumours also adapted to be used in any other natural/artificial orifices, spaces and post operative spaces.

1. A Light Irradiating Unit (LIU) for providing radiant energy safely to a treatment site within the human body during treatment sessions; wherein said LIU is comprised of the following modules:
(a) an Irradiating Array (IA) comprising a plurality of LED's longitudinally disposed in a single row along a flexible support member; said LED's are electrically connected in series to a battery powered external LED Controller and Power Supply; said IA is rotatable in situ through 360 degrees of movement;
(b) an external LED Controller and Power Supply (CPS);
(c) an Array Housing Catheter (AHC) for enclosing and guiding said IA to said treatment site; and,
(d) a Cooling Catheter (CC) for enclosing and cooling said AHC during treatment sessions; said CC is adapted to connect to a standard intra venous bag, containing cooling fluid, said bag adapted to provide gravity induced cooling fluid flow through said CC during treatment sessions; said CC further adapted to provide drainage of said cooling fluid during said treatment sessions.

2. An LIU according to claim 1 wherein said CPS further comprises a battery powered cell.

3. An LIU according to claim 1 wherein said LIU further comprises a motor driven means of rotating at least one of the group consisting of said IA, said AHC and said CC in a predetermined manner as required by the treatment protocol.

4. An LIU according to claim 1, wherein said LIU additionally comprises
(a) said CPS with a rechargeable battery
(b) first connecting means for connecting CPS to a wall power adapter for recharging said battery.
(c) second connecting means for connecting said CPS to said IA
(d) safety means adapted so as to ensure that said first or second connecting means make first or second connections respectively at any one time and that said first or second connections cannot be completed or maintained contemporaneously.

5. An LIU according to claim 1 wherein said CPS further comprises means for operation by remote control.

6. An LIU according to claim 1 wherein said CC is adapted to connect to a standard hospital pump, said pump adapted to induce cooling fluid flow during treatment sessions.

7. An LIU according to claim 1 wherein said LED's are electrically connected in parallel to a battery powered external LED CPS.

8. An LIU according to claim 1 wherein said flexible support member is fan folded.

9. A method of providing radiant energy safely to a treatment site within the human body during treatment sessions; said method including steps of:
a. obtaining an Irradiating Array (IA) and disposing a plurality of LED's longitudinally in a single row along a flexible support member; said LED's are electrically connected in series to a battery powered external LED CPS; said IA is rotatable in situ through 360 degrees of movement;
b. obtaining an external LED Controller and Power Supply (CPS);
c. obtaining an Array Housing Catheter (AHC) for enclosing and guiding said IA to said treatment site; and,
d. further obtaining a Cooling Catheter (CC) for enclosing and cooling said AHC during treatment sessions; said CC is adapted to connect to a standard intra venous bag, containing cooling fluid, adapting said bag to provide gravity induced cooling fluid flow through said CC during treatment sessions; further adapting said CC to provide drainage of said cooling fluid during said treatment sessions.

10. A method according to claim 9, wherein said method includes additional steps of powering said CPS by means of a battery cell.

11. A method according to claim 9, wherein said method further includes obtaining a motor driven means of rotating at least one of the group consisting of said IA, said AHC and said CC in a predetermined manner as required by the treatment protocol.

12. A method of providing radiant energy safely to a treatment site within the human body during treatment sessions according to claim 9, said method including additional steps of
(a) providing said CPS with a rechargeable battery
(b) firstly connecting said CPS to a wall power adapter
(c) secondly connecting said CPS to said IA
(d) adapting said first connection between said wall power adapter and said CPS, and said second connection between CPS and said IA such that only one of said first or second connections can be made at any one time,
(e) recharging said CPS by means of establishing said first connection.

13. A method according to claim 9 wherein said method includes steps of operating CPS by remote control.

14. A method according to claim 9 wherein said method includes steps of connecting said CC to a standard hospital pump, further adapting said pump to induce cooling fluid flow during treatment sessions.

15. A method according to claim 9 wherein said method includes steps of electrically connecting said LED's in parallel to a battery powered external LED CPS.

16. A method according to claim 9 wherein said method includes steps of fan folding said flexible support member.
